(19) United States
(12) Patent Application Publication
(10) Pub. No.: US 2015/0068530 A1
(43) Pub. Date: Mar. 12, 2015

(54) ONE-SIDED NASAL CANNULA DEVICE AND ATTACHMENTS

(71) Applicant: Arnaldo Apolito, Montclair, NJ (US)

(72) Inventor: Arnaldo Apolito, Montclair, NJ (US)

(21) Appl. No.: 14/019,785

(22) Filed: Sep. 6, 2013

(51) Int. Cl. A61M 16/06

(52) U.S. Cl. CPC A61M 16/0666; A61M 16/0683
USPC 128/207.18

(57) ABSTRACT

A nasal cannula device for delivering therapeutic gas to a patient while reducing rubbing, scraping or other damage or discomfort is disclosed. In an aspect, a nasal cannula device is configured to removably attach to a patient via a single ear and comprises gas tubing and an anchor. The gas tubing comprises a gas source connector, a gas tubing body, and a nasal member. The anchor attaches to the gas tubing body and is removably positionable over a patient ear. In an aspect, the nasal member further comprises at least one nasal insert device configured to retain the nasal member in a patient nostril and reduce impacts and discomfort via a compressible member. The compressible member may form an airtight barrier, thereby protecting a patient nasal airway from environmental contaminants.

### FIELD OF THE DISCLOSURE

The present disclosure relates to medical devices and more particularly cannula devices for delivering supplemental oxygen.

### BACKGROUND

The statements in this section merely provide background information related to the present disclosure and may not constitute prior art.

Numerous patients require respiratory assistance such as the delivery of supplemental oxygen. Oxygen is normally delivered to the patient through oxygen delivery devices such as nasal cannula or masks. The nasal cannula is often preferred over the mask because it does not interfere with the ability to communicate, eat or drink. Those requiring use of the nasal cannula often include elderly patients. The nasal cannula may also be used by pilots and passengers in small unpressurized aircraft that don't exceed certain altitudes.

The existing nasal cannula device comprises a first nasal tubing and a second nasal tubing. Each nasal tubing has a first end connected to a gas source and a second prong end configured to be placed within a patient's nostril. Each nasal tubing is placed over a patient ear, thereby providing a removable stabilizing connection for the nasal cannula via the patient's two ears. Such cannula devices may provide therapeutic gas to the patient. For example, pure oxygen; a mixture of oxygen and nitrogen; a nitrous oxide mixture; or other gases may be provided.

Gas may flow freely out of the prong end portions of the cannula device positioned within the patient's nostrils. This gas flow may continue even when the patient is not inhaling, causing the therapeutic gas being provided to flow out of the patient's nostrils where the gas is lost. Furthermore, this lost gas may irritate the patient's eyes.

Current cannula devices may cause significant rubbing, cutting, scabbing, and irritation where the cannula device makes contact with the patient's skin. This is especially problematic for elderly patients because such patients have a reduced ability to heal.

As previously described, the current nasal cannula devices normally attach to the patient by each nasal tubing coming from the nose and hooking around the ears on both sides of the face and thereafter connecting under the chin by a sliding connector configured to hold each nasal tubing in place . The first end of each nasal tubing may then be connected to a mobile or static gas source. For those patients who desire to sleep on their side, the nasal cannula often digs into the side of the face, leaving unsightly marks or, in some cases, causing bruising or scraping the skin. In this position, the ear being slept upon is sandwiched between the nasal tubing positioned above the ear and the sleeping surface, causing discomfort and disrupting blood flow to both the ear lobe. Where the nasal tubing is flexible, the nasal tubing may be pinched, impeding the flow of therapeutic gas to the patient. The described discomfort and other considerations forces the patient to sleep on their back to avoid such injuries. Such considerations are not limited to sleep positions. Any situation which causes the patient to place something against one side of their head or an ear (e.g., wearing headphones) may cause similar discomfort.

To further complicate matters, many patients find that the tube often gets stuck around items such as a protruding piece of furniture, a door knob, a door itself, the wearer's feet or even the feet of someone within close proximity. When a portion of a current cannula device is caught on an object, not only does the nasal tubing painfully pull against the patient's ear, the prong end portions may scrape or uncomfortably impact the sensitive inner portions of the patient's nose.

Some patients requiring supplementary oxygen may be instructed to minimize their exposure to environmental contaminants such as dust, bacteria, viruses and pollens. Such patients include those suffering from Chronic Obstructive Pulmonary Disease (COPD). In these circumstances, oxygen masks have traditionally been utilized because they reduce exposure of the patient's airways to the surrounding environment and the associated contaminants. The oxygen face mask is a plastic oxygen delivery device that is shaped to fit over a patient's nose and mouth. The mask contains open slits which allow air from the room to enter the mask and dilute the oxygen, while allowing exhaled carbon dioxide to leave the restrained space. Like the nasal cannula, plastic tubing is fixed to the bottom of the mask and connected to an oxygen source. The mask is held in place by an elastic band which goes over the patient's head and above the patient's ears to hold the mask in place. The masks pose great difficulty in performing everyday tasks such as talking and eating, as well as being cosmetically unattractive.

Given the foregoing, what is needed is a nasal cannula device capable of delivering therapeutic gases while reducing rubbing, scraping, and other damage or discomfort to the patient during, especially during long term use. Additionally, cannula devices are desired which inhibit the patient's movement less than current cannula devices. A cannula device which minimizes exposure of the patient's nasal airways to the outside environment is also desired.

Additionally, what is needed is a cannula device which does not cause the patient to deviation from their preferred sleep position (i.e., forcing the patient to sleep on their back when they may prefer to sleep on their side).

### SUMMARY

This Summary is provided to introduce a selection of concepts in a simplified form that are further described below in the Detailed Description. This Summary is not intended to identify key features or essential features of the subject matter, nor is it intended to be used to limit the scope of the subject matter.

This disclosure addresses the above-described needs by providing nasal cannula devices configured to deliver therapeutic gases (e.g., supplemental oxygen) to a patient via a one-sided device which reduces rubbing, scraping, or other damage or discomfort to the patient. Such devices may enable greater range of motion for the patient, allow easier access to portions of the patient's face, ear, and neck not engaged by the device, and enable the patient to sleep in their preferred sleep position (e.g., on their side).

In an aspect, a nasal cannula device removably attaches to a patient's body on one side of the head and comprises an anchor and gas tubing. The gas tubing comprises a gas source connector, a gas tubing body, and a nasal member. The gas source connector is configured to engage a gas source (e.g., mobile oxygen canister) and enable the flow of gas into the remaining portions of the nasal cannula device. The gas tubing body may be a flexible, rigid, or semi-rigid length of tubing configured to transport gas received at the gas source connector to the patient's nasal airways via the nasal member. The nasal member may be a rigid member comprising a first nasal member end portion and two nasal interfaces. The first nasal member end portion in configured to receive gas from the gas tubing. Each nasal interface is configured to removably interface with a patient nostril and provide therapeutic gas to the patient nostril. The nasal interfaces may be rigid, parallel tubes configured to be inserted into the corresponding patient nostril. Such nasal interfaces may be connected by a rigid tube, thereby separating them a distance from one another. This separation distance may be chosen in order to position each nasal interface within each patient nostril.

The anchor is attached to the gas tubing at the gas tubing body and is adapted to removably connect the nasal cannula device to a patient ear. The anchor may be a curved member extending over the patient ear and attaching to the gas tubing body at an anchor end portion. The anchor end portion may slidably, removably and/or rotatably connect with the gas tubing body, enabling the patient to adjust the position of the gas tubing body in order to increase their comfort level while wearing the device. A rotatable connector also enables the patient to switch the ear over which the anchor hangs, allowing the patient to sleep on a preferred side or otherwise leave a preferred ear unencumbered.

In other aspects, the anchor is configured to interface with the patient ear in a different manner.

In an aspect, a nasal cannula device removably attaches to the patient's head via an over the scalp connector.

Other aspects of the present disclosure provide attachments for a nasal cannula device which minimizes exposure of the patient's nasal airways to the outside environment. In such aspects of the present disclosure, the traditional prongs of existing nasal cannula devices may be replaced by two compressible members, such as foam hemispheres, sealed with a filtering element. The compressible member may comprise a gas passageway which connects to the cannula and allows therapeutic gas to enter the patient's nostril. The compressible members may be configured to snugly, removably fit into the nostrils, thereby providing a comfortable connection for the cannula which preventing unfiltered air and environmental contaminants to enter the patient's body via the nostrils.

In an aspect, nasal cannula attachments may be utilized with one-sided nasal cannula devices disclosed herein.

Further features and advantages of the present disclosure, as well as the structure and operation of various aspects of the present disclosure, are described in detail below with reference to the accompanying drawings.

### BRIEF DESCRIPTION OF THE DRAWINGS

The features and advantages of the present disclosure will become more apparent from the Detailed Description set forth below when taken in conjunction with the drawings in which like reference numbers indicate identical or functionally similar elements.

FIG. 1 is a front view of a one-sided nasal cannula device being worn by a patient, in accordance with an aspect of the present disclosure.

FIG. 2 is a front view of a one-sided nasal cannula device, depicting an alternative configuration of the anchor, in accordance with an aspect of the present disclosure.

FIG. 3 is a front view of a one-sided nasal cannula device being worn by a patient on an alternate side, in accordance with an aspect of the present disclosure.

FIG. 4 is a front view of a one-sided nasal cannula device being worn by a patient on an alternate side, wherein the gas tubing of the one-sided nasal cannula device is also configured to anchor the device, in accordance with an aspect of the present disclosure.

FIG. 5 is a side view of a one-sided nasal cannula device being worn by a patient, in accordance with an aspect of the present disclosure.

FIG. 6 is a side view of a one-sided nasal cannula device being worn by a patient, wherein the anchor end portion comprises a swivel, in accordance with an aspect of the present disclosure.

FIG. 7 is a side view of a one-sided nasal cannula device being worn by a patient, in accordance with an aspect of the present disclosure.

FIG. 8 is a front view of a one-sided nasal cannula device, wherein the cannula device comprises a secondary anchor, in accordance with an aspect of the present disclosure.

FIG. 9 is a front view of a one-sided nasal cannula device, wherein the anchor is configured to be placed on top of the patient head, in accordance with an aspect of the present disclosure.

FIG. 10 is a of a one-sided nasal cannula device, depicting an alternative configuration of the anchor, in accordance with an aspect of the present disclosure.

FIG. 11 is a front view of a nasal member equipped with two nasal inserts, in accordance with an aspect of the present disclosure.

FIG. 12 is a top view of a nasal member equipped with two nasal inserts, in accordance with an aspect of the present disclosure.

FIG. 13 is a front view of a nasal member equipped with two nasal inserts, depicting an alternative configuration, in accordance with an aspect of the present disclosure.

FIG. 14 is a detail view of a nasal member equipped with two nasal inserts, shown inserted into patient nostrils, in accordance with an aspect of the present disclosure.

FIG. 15 is a front view of a nasal cannula equipped with two nasal inserts being worn by a patient, in accordance with an aspect of the present disclosure.

FIG. 16 is a front view of a one-sided nasal cannula device equipped with two nasal inserts being worn by a patient, in accordance with an aspect of the present disclosure.

### DETAILED DESCRIPTION

The present disclosure is directed to devices and attachments which allow for delivery of therapeutic gases (e.g., pure oxygen; a mixture of oxygen and nitrogen; a nitrous oxide mixture; other gases and the like) to a patient via a one-sided device which reduces rubbing, scraping, or other damage or discomfort to the patient. Such devices may enable greater range of motion for the patient, allow easier access to portions of the patient's face, ear, and neck not engaged by the device, and enable the patient to sleep in their preferred sleep position (e.g., on their side).

In an aspect, a nasal cannula device removably attaches to a patient's body on one side of the head and comprises an anchor and gas tubing.

Devices in accordance with the present disclosure may be utilized with nasal cannula devices in order to minimize exposure of the patient's nasal airways to the outside environment.

Unless otherwise noted, for the purposes of the present disclosure, “patient” and its plural forms refers to humans, animals, and other living creatures which may receive therapeutic gases using devices disclosed herein. In particular, patient includes individuals seeking medical care, individuals under long term care, pilots, athletes, horses, dogs, other mammals, and the like.

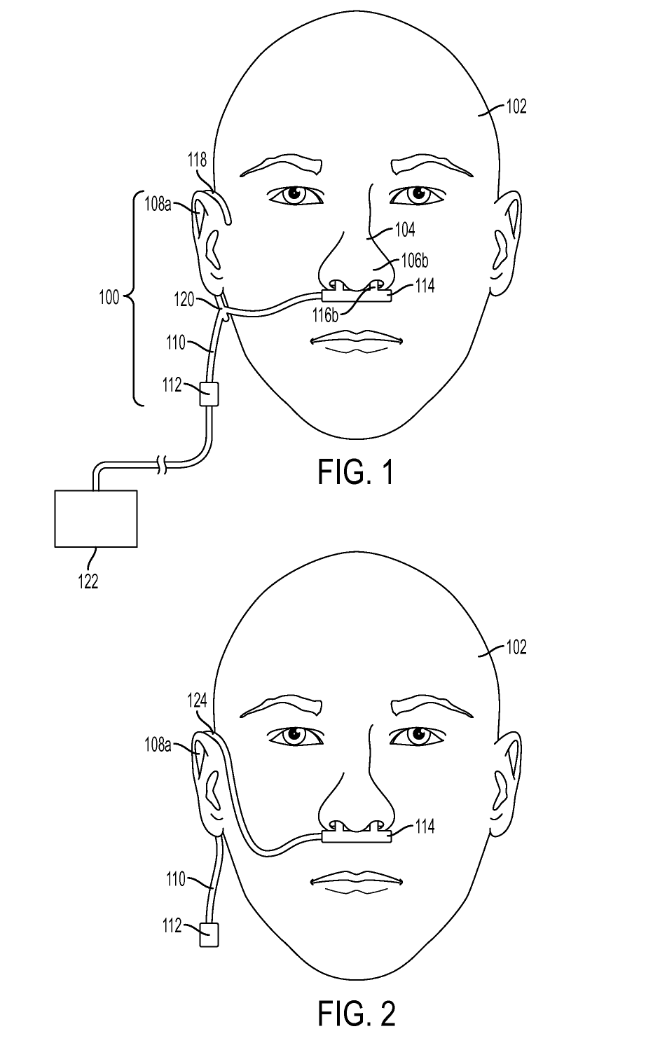

Referring now to FIG. 1, a front view of a one-sided nasal cannula device 100 being worn by a patient 102, in accordance with an aspect of the present disclosure, is shown.

Nasal cannula device 100 removably attaches to a patient's body on one side of the patient head (e.g., at an ear 108a (labeled, for clarity, only as ear 108a in FIG. 1)) and comprises an anchor 118 and gas tubing 110.

Gas tubing 102 is configured to deliver therapeutic gases received from gas source 122 to a patient airway via a patient nose 104. Gas tubing 110 may comprise a gas source connector 112, a gas tubing body, and a nasal member 114.

Gas source connector 112 is configured to engage gas source 122 (e.g., a portable oxygen tank, wall supply source in the hospital setting, and the like) and enable the flow of gas into the remaining portions of nasal cannula device 100. Gas source connector 112 may be a permanent connector, a screw-on connector, a removable connector, a quick-release connector, or any other connection means apparent to those having skill in the relevant art(s) after reading the description herein.

The gas tubing body may be a flexible, rigid, or semi-rigid length of tubing configured to transport gas received at gas source connector 112 to the patient's nasal airways via nasal member 114. Tubing may be the diameter or standard nasal cannula tubing. In another aspect, gas tubing body may have an increased diameter, in order to deliver a therapeutic volume of gas at a therapeutic rate. The gas tubing body maybe be several feet long to only a few inches, depending on application.

In another aspect, the gas tubing body is an expandable bladder or other member configured to transport gas received at gas source connector 112 to the patient's nasal airways via nasal member 114.

Nasal member 114 may be a rigid member comprising a first nasal member end portion and two nasal interfaces a (labeled, for clarity, only as nasal interface 116b in FIG. 1.). The first nasal member end portion in configured to receive gas from the remainder of gas tubing. Each nasal interface 116 is configured to removably interface with a patient nostril and provide therapeutic gas to the patient nostril. Nasal interface 116 may be rigid, parallel tubes configured to be inserted into the corresponding patient nostril. Such nasal interface 116 may be connected by a rigid tube, thereby separating them a distance from one another. This separation distance may be chosen in order to position each nasal interface 116 within each patient nostril.

Anchor 118 is attached to gas tubing 110 at, for example, the gas tubing body. Anchor 118 is adapted to removably connect nasal cannula device 100 to a single patient ear 108. Anchor may be a curved member extending over patient ear 108 and attaching to gas tubing 110 at an anchor end portion 120. Anchor end portion 120 may slidably, removably and/or rotatably connect with gas tubing 110, enabling patient 102 to adjust the position of gas tubing 110 in order to, for example, increase their comfort level while wearing nasal cannula device 100.

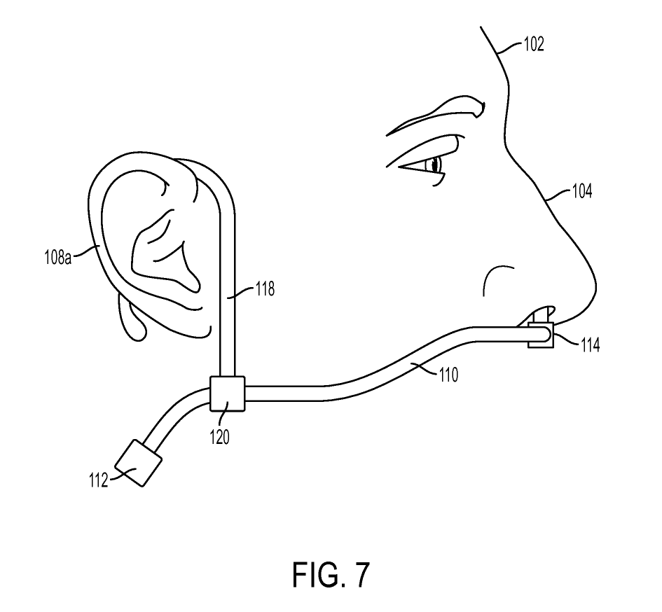

Anchor 118 may be at least partially constructed of polymer, ABS plastic, carbon fiber, metal, rubber, or another durable material. Anchor 118 may loop over patient ear 108 and extend down behind patient ear 118 in order to connect with gas tubing 110 at anchor end portion 120. In another aspect (FIG. 7), anchor loops over patient ear 108 and extends down in front of patient ear 118 in order to connect with gas tubing 110 at anchor end portion 120.

In another aspect, anchor 118 may comprise a flexible material that may be secured over patient ear 108.

Anchor end portion 120 may be a permanent or removable connection to gas tubing 110. Anchor end portion 120 may comprise a sliding connector assembly or other removable connection means as will be apparent to those skilled in the relevant art(s) after reading the description herein.

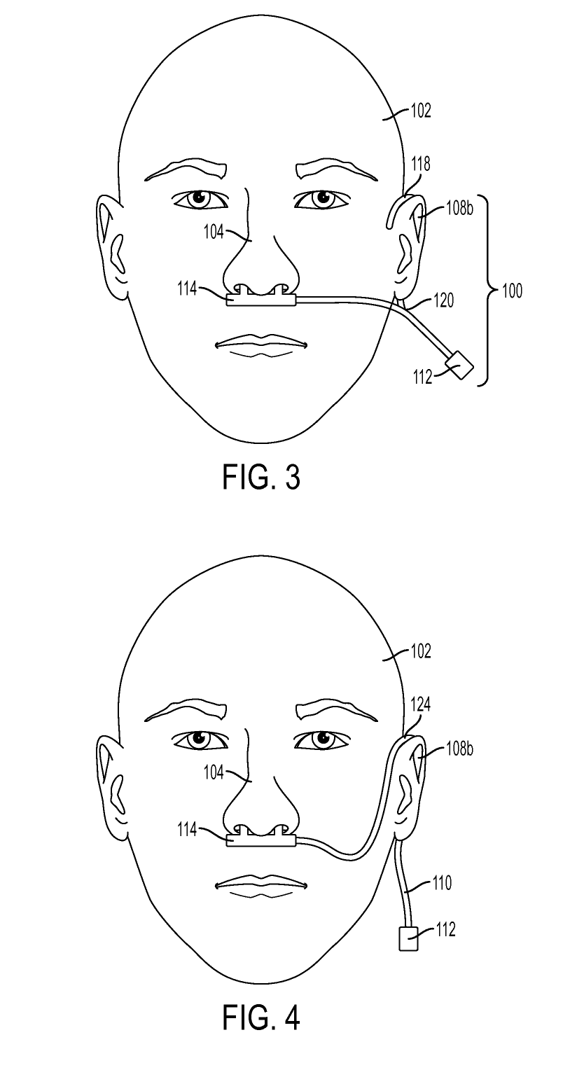

In some aspects, patient 102 may position nasal cannula device 100 on either side of the face. For example, as shown in FIGS. 3 & 4, if patient 102 prefers to sleep on the right side of his/her body, patient may choose to secure nasal cannula device 100 on the left side of the face via placing anchor 118 on left patient ear 108b so as not to disturb patient 102 while sleeping. Similarly, if patient 102 prefers to sleep on the left side of his/her body, patient 102 may choose to secure nasal cannula device 100 on the right side of the face via placing anchor 118 over right patient ear 108a so as not to disturb the patient 102 while sleeping. The preference of patient 102 may be taken into account at any time and is not limited to the time in which patient 102 is sleeping.

In some aspects, patient 102 may choose to wear nasal cannula device 100 under his/her clothing on either side of his/her body so as to prevent patient 102 from tripping over a portion of device 100. In other aspects, patient 102 may choose to wear nasal cannula device 100 under his/her clothing so as to prevent a portion of device 100 from getting caught on items such as a door, door knob, table, or the like.

Nasal cannula device 100 may be a lightweight device constructed of one or more materials such plastic, silicone or the like.

Referring now to FIG. 2, a front view of one-sided nasal cannula device 100, depicting an alternative configuration of anchor 118, in accordance with an aspect of the present disclosure, is shown.

In an aspect, nasal cannula device 100 comprises gas tubing 110. Gas tubing 110 comprises gas source connector 112, gas tubing body, and nasal member 114. Gas tubing body comprises formed member 124. Formed member 124 is a portion of gas tubing body which is rigid or semi-rigid (i.e., having some flexibility or ability to be formed into a shape by patient 102 or a third party) having a shape which facilitates placement over patient ear 108, thereby retaining nasal cannula device 100.

In an aspect, as least a portion of nasal cannula device 100 is flexible. This flexibility facilitates, for example: placement and positioning of nasal member 114; placing nasal cannula device 100 on either patient ear 108; and the like. Some or all of gas tubing 110 may be flexible including, but not limited to portions of gas tubing body.

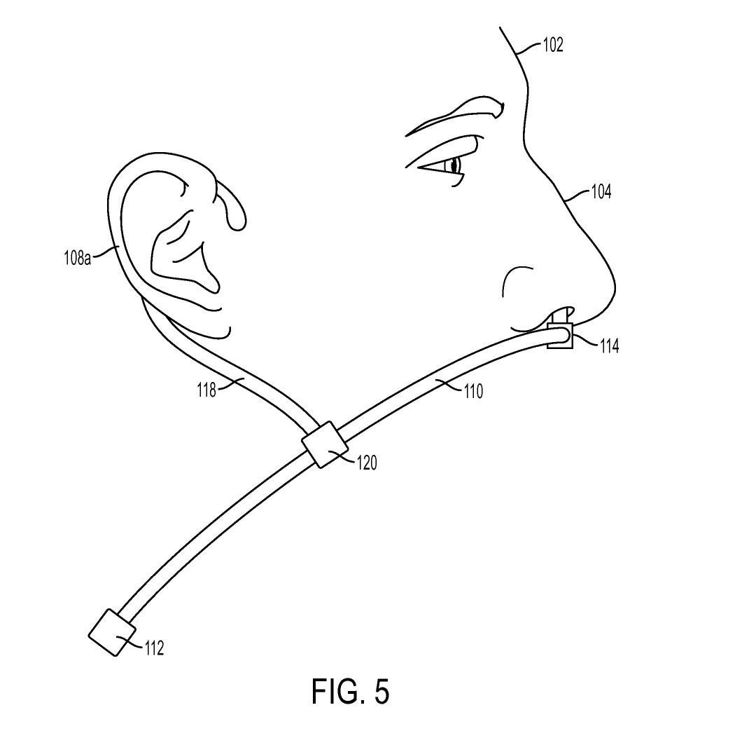

Referring now to FIG. 5, a side view of one-sided nasal cannula device 100 being worn by patient 102, in accordance with an aspect of the present disclosure, is shown.

Anchor 118 may be designed with varying heights, widths, and cross sections. In an aspect, anchor 118 may be custom designed for patient 102. In another aspect, anchor may be configured to break away from gas tubing 110 at, for example, anchor end portion 120 in the even that gas tubing snags on an object, thereby avoiding tugging on patient ear 108 or nose 104.

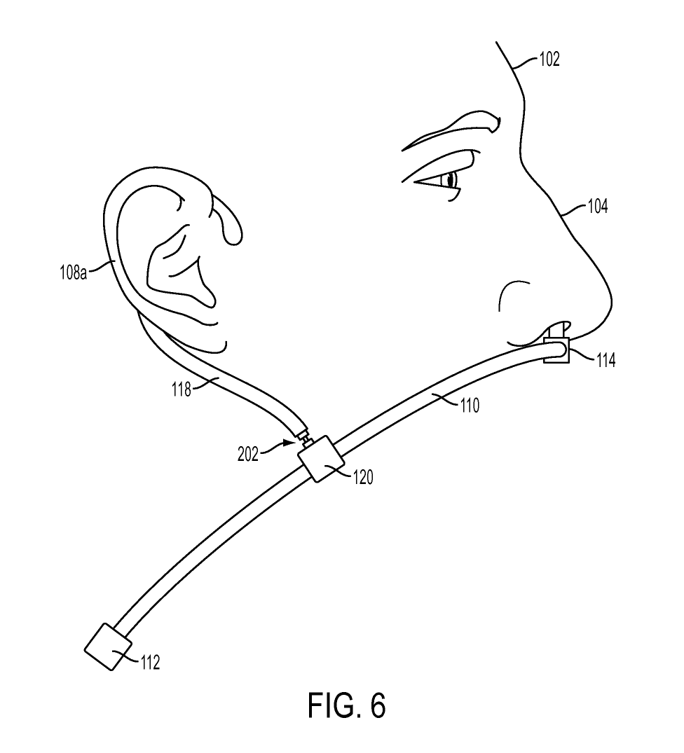

Referring now to FIG. 6, a side view of one-sided nasal cannula device 100 being worn by patient 102, wherein anchor end portion 120 comprises a swivel 202, in accordance with an aspect of the present disclosure, is shown.

In an aspect, anchor end portion 120 may comprise swivel 202 or other movable member. Swivel 202 facilitates placement of nasal cannula device 100 during use.

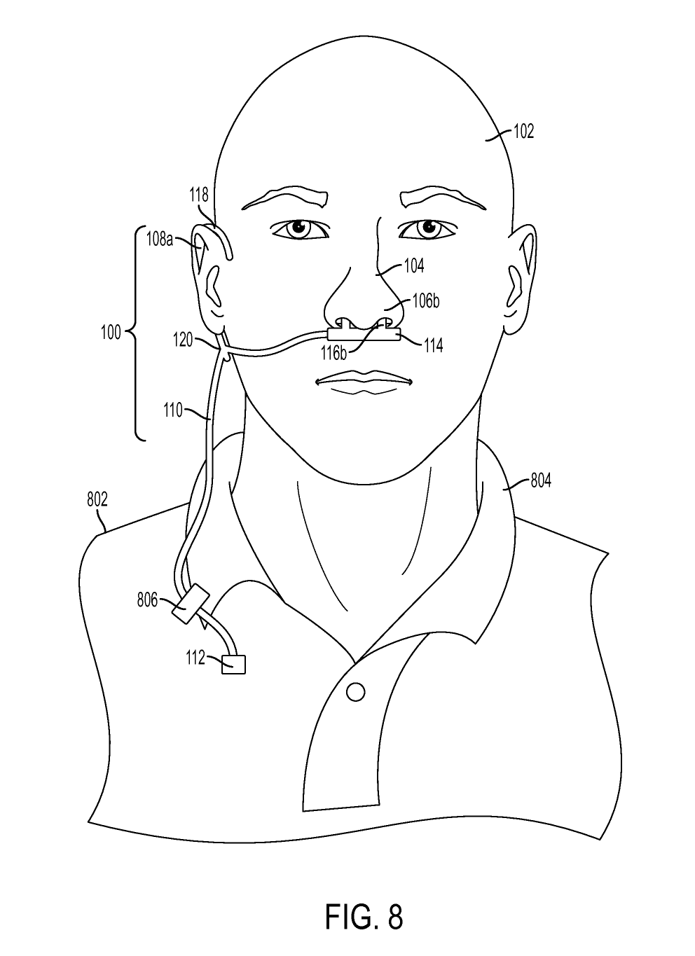

Referring now to FIG. 8, a front view of a one-sided nasal cannula device, wherein the cannula device comprises a secondary anchor 806, in accordance with an aspect of the present disclosure, is shown.

Secondary anchor 806 may be configured to removably connect nasal cannula device 100 to patient clothing 802. In an aspect, secondary anchor 806 is a clip which removably connects gas tubing 110 to collar 804.

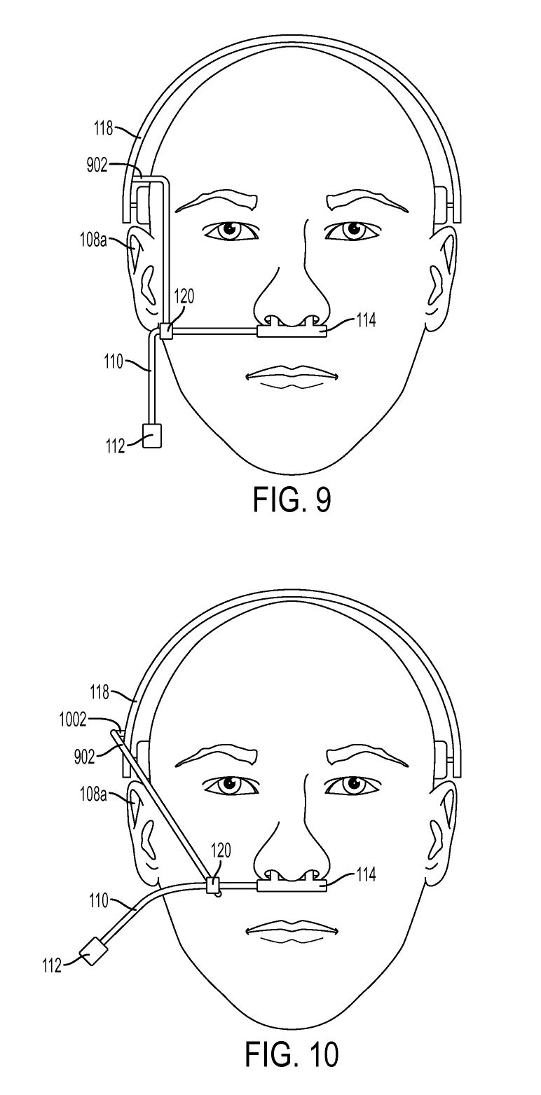

Referring now to FIG. 9, a front view of one-sided nasal cannula device 100, wherein anchor 118 is configured to be placed on top of the head of patient 102, in accordance with an aspect of the present disclosure, is shown.

In an aspect, anchor 118, similar to the brace of a unidirectional headset or a headset microphone worn by disc jockey, is configured to pass over the head of patient 102. Anchor 118 comprises boom 902. Boom 902 may be a rigid or flexible member which extends to a position where anchor end portion 120 connects to gas tubing 110. As shown in FIG. 10, anchor 118 may further comprise pivot 1002, thereby allowing boom 902 to be moved to a desired position during use.

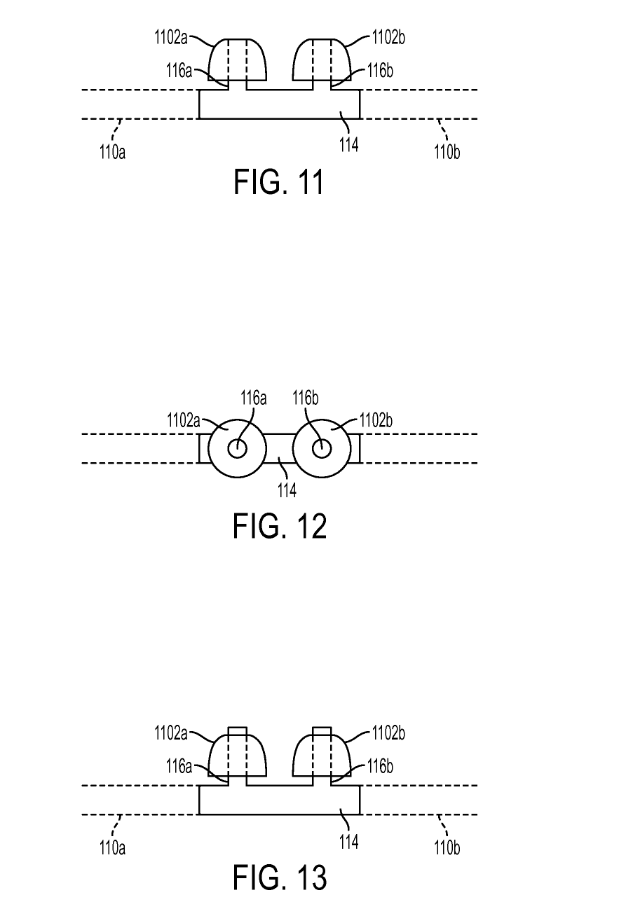

Referring now to FIG. 11, a front view of nasal member 114 equipped with two nasal inserts 1102 (labeled as nasal inserts 1102a & b in FIG. 11), in accordance with an aspect of the present disclosure, is shown.

Nasal insert 1102 is a compressible member configured to be removably inserted into patient nose 104 at a nostril of patient 108. Nasal insert 1102 may be constructed of foam, rubber, latex, sponge, “memory” foam, or another deformable or flexible material apparent to those having skill in the relevant art(s) after reading the description herein. Nasal insert 1102 may be configured to serve one or more purposes including: forming a barrier between the nasal airway of patient 108 and the surrounding environment; facilitating retaining nasal member 114 in a position where therapeutic gases may be delivered to the nasal airway; and protecting the inner surface of nose 104 from impacts with nasal interface 116 (labeled as nasal interfaces 116a & b in FIG. 11).

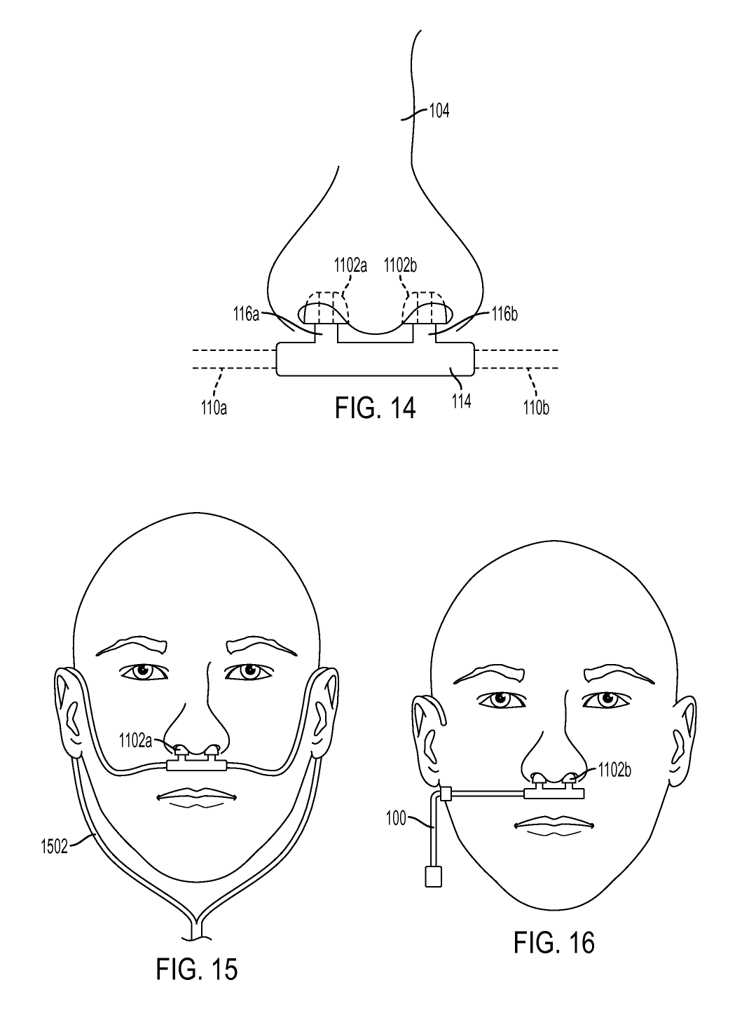

As shown in FIGS. 11 and 12, in an aspect, nasal insert 1102 is a hemisphere of foam material having a cylindrical conduit through its axis. The diameter of the cylindrical conduit may be equal to the outer diameter of the nasal interface 116 of nasal cannula devices, thereby enabling the placement of nasal insert 1102 over nasal interface 116. Nasal interfaces 116 equipped in this manner may then be inserted into the nostril, as shown in FIG. 14.

In another aspect, nasal insert 1102 comprises a cylindrical conduit through its vertical axis configured to removably receive nasal interface 116. The outer shape of nasal insert 1102 may be a portion of an ellipsis, a cylinder, or another shape which loosely or tightly conforms to the inner region of a nostril of nose 104.

In an aspect where nasal insert 1102 provides a barrier to environmental contaminants, nasal insert 1102 may comprise a flared end in order to facilitate creation of an air tight barrier to the nostril. In another such aspect, nasal insert 1102 may form an air tight or near air tight barrier via the shape of nasal insert 1102, via expansion of nasal interface 1102 within the nostril, via comprising an air impermeable bottom surface, or via another means apparent to those having skill in the relevant art(s) after reading the description herein.

Nasal insert 1102 may be semipermeable. In an aspect, nasal insert 1102 may, for example, be configured to allow carbon dioxide to exit the nostril. In another aspect, nasal insert 1102 may be at least partially constructed of a filtering material which allows purified air from the surrounding environment to enter the nostril.

In an aspect, nasal insert 1102 is constructed of a deformable, compressible material. In order to wear nasal member 114 equipped with nasal inserts 1102, patient 102 compresses each nasal insert 1102 along its vertical axis and inserts the nasal insert-equipped nasal interfaces 116 into each nostril of nose 104. Each nasal insert 1102 may then slowly expand until reaching the inner walls of the nostril. Nasal insert 1102 may be constructed such that the static perimeter of nasal insert 1102 is larger than the perimeter of the inside of the nostril. In this manner, nasal insert 1102 may press against the inner walls of the nostril, providing a retaining force and protecting the inner walls from impact with nasal interface 116.

As shown in FIG. 14, nasal insert 1102 may be configured to cover only a portion of nasal interface 116.

Nasal insert 1102 may be removably attached to nasal member 114 or permanently attached via, for example, adhering nasal insert 1102 to nasal interface 116.

Referring now to FIGS. 15 & 16, front views of nasal inserts 1102 being utilized with one-sided nasal cannula device 100 and a two-sided nasal cannula device, in accordance with aspects of the present disclosure, are shown.

Nasal insert 1102 may be used with a variety of styles of nasal cannula devices now know or developed in the future.

While various aspects of the present disclosure have been described above, it should be understood that they have been presented by way of example and not limitation. It will be apparent to persons skilled in the relevant art(s) that various changes in form and detail can be made therein without departing from the spirit and scope of the present disclosure. Thus, the present disclosure should not be limited by any of the above described exemplary aspects, but should be defined only in accordance with the following claims and their equivalents.

In addition, it should be understood that the figures in the attachments, which highlight the structure, methodology, functionality and advantages of the present disclosure, are presented for example purposes only. The present disclosure is sufficiently flexible and configurable, such that it may be implemented in ways other than that shown in the accompanying figures. As will be appreciated by those skilled in the relevant art(s) after reading the description herein, certain features from different aspects of the devices and systems of the present disclosure may be combined to form yet new aspects of the present disclosure.

Further, the purpose of the foregoing Abstract is to enable the U.S. Patent and Trademark Office and the public generally and especially the scientists, engineers and practitioners in the relevant art(s) who are not familiar with patent or legal terms or phraseology, to determine quickly from a cursory inspection the nature and essence of this technical disclosure. The Abstract is not intended to be limiting as to the scope of the present disclosure in any way.

1. A nasal cannula device for delivering at least one therapeutic gas to a patient, comprising:
a gas tubing for delivering the at least one therapeutic gas to the patient via a nasal airway of the patient, the gas tubing comprising:
a gas source connector configured to engage a gas source and receive the at least one therapeutic gas from the gas source;
a gas tubing body connected to the gas source connector for transportation of the at least one therapeutic gas; and
a nasal member connected at a first nasal member end portion to the gas tubing body for transporting the at least one therapeutic gas into the nasal airway of the patient via a nasal interface, the nasal interface removably insertable into a first patient nostril and a second patient nostril; and
a single anchor attached at an anchor end portion to the gas tubing at the gas tubing body, the anchor formed to be removably positioned over a first patient ear;
wherein the anchor is selectably positionable on a patient right ear or a patient left ear; and
wherein, during wear of the nasal cannula device, a second patient ear is not contacted by the nasal cannula device.

2. The nasal cannula device of claim 1, wherein:
the anchor is a curved member extending over an ear of the patient; and
the anchor end portion extends behind the ear of the patient.

3. The nasal cannula device of claim 1, wherein:
the anchor is a curved member extending over an ear of the patient; and
the anchor end portion extends in front of the ear of the patient.

4. The nasal cannula device of claim 1, wherein the anchor may be selectably positioned over a right ear of the patient and over the left ear of the patient.

5. The nasal cannula device of claim 1, wherein the anchor passes over the head of the patient and removably contacts both sides of the head of the patient, the anchor further comprising:
a boom;
wherein a distal end of the boom is the anchor end portion;
wherein the boom is positioned to attach to the gas tubing.

6. The nasal cannula device of claim 1, wherein the gas tubing is removably connected to the anchor.

7. The nasal cannula device of claim 1, wherein the gas tubing is rotatably connected to the anchor.

8. The nasal cannula device of claim 1, further comprising:
a secondary anchor attached to the gas tubing at the gas tubing body configured to removably attach to an article of clothing of the patient.

9. The nasal cannula device of claim 1, the nasal member further comprising:
a second nasal interface, the second nasal interface removably insertable into a second nostril of the patient.

10. A nasal cannula device, comprising:
a gas tubing for delivering a gas to a patient via a nasal airway of the patient, the gas tubing comprising:
a gas source connector configured to engage a gas source and receive the gas from the gas source;
a gas tubing body connected to the gas source connector for transportation of the gas, including a body portion configured to be removably positioned over a first ear of the patient for stabilization, the body portion having a soft exterior surface;
a nasal member connected at a first nasal member end portion to the gas tubing body for transportation of the gas into the nasal airway of the patient via a nasal interface having a pair of nasal inserts, the pair of nasal inserts removably insertable into nostrils of the patient; and
a pair of compressible members positioned over the pair of nasal inserts, each compressible member configured to be removably inserted into a single nostril of the patient, each compressible member having a cylindrical conduit running the length of the compressible member, having an uncompressed width greater than an inner nostril width;
wherein, after insertion into the single patient nostril, each compressible member expands against an inner nostril surface, exerting a retaining force;
wherein each compressible member at least partially comprises a filtering material which purifies the air from the surrounding environment that enters the single nostril;
wherein the compressible member is semipermeable to gases and configured to allow nasal exhalation; and
wherein, during wear of the nasal cannula device, a second ear of the patient is not contacted by the nasal cannula device.

11. The nasal cannula device of claim 10, wherein the formed member portion is selectably positioned over a right ear of the patient and a left ear of the patient.

12. The nasal cannula device of claim 1, the nasal member further comprising:
a second nasal interface, the second nasal interface removably insertable into a second nostril of the patient.

13. A nasal insert device for improving the functionality of a nasal cannula device, comprising:
a hemispherically shaped compressible member configured to be removably inserted into a nostril of a patient, the compressible member having a cylindrical conduit running the length of the compressible member, having an uncompressed width greater than an inner nostril width;
wherein, after insertion into the nostril of the patient, the compressible member expands against an inner nostril surface, exerting a retaining force; and
wherein a nasal interface may be inserted into the cylindrical conduit and provide therapeutic gas to the patient via the nostril of the patient.

14. The nasal insert device of claim 13, wherein the compressible member is a hemisphere.

15. The nasal insert device of claim 13, wherein the compressible member is at least partially constructed of foam.

16. The nasal insert device of claim 13, wherein, the compressible member forms an airtight barrier when inserted into the nostril of the patient.

17. The nasal insert device of claim 13, wherein the compressible member is configured to reduce impacts between the inner nostril surface and the nasal interface inserted into the cylindrical conduit.

18. The nasal insert device of claim 13, wherein the compressible member is configured to filter impurities from the surrounding environment.

19. The nasal insert device of claim 13, wherein the compressible member is semipermeable to and configured to allow nasal exhalation.

20. A nasal cannula device for delivering at least one therapeutic gas to a patient, comprising:
a gas tubing for delivering the at least one therapeutic gas to the patient via a nasal airway of the patient, the gas tubing comprising:
a gas source connector configured to engage a gas source and receive the at least one therapeutic gas from the gas source;
a gas tubing body connected to the gas source connector for transporting the at least one therapeutic gas;
a nasal member connected at a first nasal member end portion to the gas tubing body for transporting the at least one therapeutic gas into the nasal airway of the patient via a first nasal interface, the first nasal interface removably insertable into a first nostril of the patient and via a second nasal interface, the second nasal interface removably insertable into a second nostril of the patient; and
an anchor attached at an anchor end portion to the gas tubing at the gas tubing body, the anchor being an inverted U-shaped member formed to be selectably positioned over a single first patient ear, the first patient ear being one of: a right ear of the patient and over a left ear of the patient;
wherein the anchor end portion extends below the ear of the patient;
wherein the anchor end portion comprises a swivel;
wherein the gas tubing is removably connected to the anchor;
wherein the gas tubing is constructed at least partially of plastic; and
wherein each of: the first nasal interface and the second nasal interface further comprises a nasal insert, the nasal insert comprising:
a compressible member configured to be removably inserted into a nostril of the patient, the compressible member having a cylindrical conduit running the length of the compressible member, having an uncompressed width greater than an inner nostril width;
wherein, after insertion into the patient nostril, the compressible member expands against an inner nostril surface, exerting a retaining force;
wherein the compressible member at least partially comprises a filtering material which purifies the air from the surrounding environment that enters the nostril;
wherein the compressible member is semipermeable to gases and configured to allow nasal exhalation;
wherein the compressible member is hemispherically shaped; and
wherein, during wear of the nasal cannula device, a single second patient ear is not contacted by the nasal cannula device.
